(19) United States
(12) Plant Patent Application Publication
(10) Pub. No.: US 2018/0192559 P1
(43) Pub. Date: Jul. 5, 2018

(54) Vaccinium corymbosum L. plant named 'RYOKU NH-13'

(50) ;

(71) Applicant: NIPPON RYOKUSAN CO., LTD., Matsumoto-shi (JP)

(72) Inventor: Shigetaka Sakurai, Matsumoto-shi (JP)

(73) Assignee: NIPPON RYOKUSAN CO., LTD., Matsumoto-shi (JP)

(21) Appl. No.: 15/732,726

(22) Filed: Dec. 21, 2017

(30) Foreign Application Priority Data
Jan. 4, 2017 (JP) 31725

(51) Int. Cl. A01H 5/08

(52) U.S. Cl. PLT/157

(57) ABSTRACT

A new and distinct variety of Vaccinium corymbosum L. plant named ‘RYOKU NH-13’, characterized by having a more upright plant growth habit, stronger plant vigor, comparatively early fruit ripening time, comparatively large fruit size, sweeter fruits, better taste balance of sweetness and acidity, better taste of fruits, and smaller stem scar, as compared to other Vaccinium corymbosum L. varieties.

The Latin name of the genus and species of the novel variety disclosed herein is: Vaccinium corymbosum L.

The novel variety of the Vaccinium corymbosum L. disclosed herein has been given the variety denomination: ‘RYOKU NH-13’.

### CROSS-REFERENCE TO RELATED APPLICATIONS

This application claims the benefit of Japanese Plant Breeders' Rights Application No. 31725, filed Jan. 4, 2017, which is incorporated by reference herein as if set forth in its entirety.

### BACKGROUND OF THE INVENTION

The present invention relates to a new and distinct hybrid variety of northern highbush blueberry (Vaccinium corymbosum L.) named ‘RYOKU NH-13’. This novel variety was found by open pollination of ‘Chandler’, a seed parent variety, in the tests conducted for the period from 2003 to 2007 in Matsumoto-City, Nagano-prefecture, Japan. As stated below, ‘RYOKU NH-13’ has apparently different characteristics from those of the varieties ‘Chandler’ and ‘Blueray’, both being widely planted and being important varieties in the Chubu district of Japan.

### SUMMARY OF THE INVENTION

Blueberry variety ‘RYOKU NH-13’ exhibits outstanding and distinguishing characteristics when grown under normal horticultural conditions in the area from Nagano-prefecture to the north of the Kanto in Japan, including:
(1) more upright plant growth habit and stronger plant vigor;
(2) comparatively early fruit ripening time (on average, around July 10 of each year in Matsumoto, Nagano, Japan);
(3) comparatively large fruit size;
(4) sweeter fruits;
(5) better taste balance of sweetness and acidity;
(6) better taste of fruits; and
(7) smaller stem scar.

### BRIEF DESCRIPTION OF THE DRAWINGS

The accompanying colored photographs (FIGS. 1-8) show typical bush, flower, fruit and leaf characteristics for the new Vaccinium corymbosum L. plant, ‘RYOKU NH-13’. Colors shown are as accurate as can be reasonably reproduced by photographic means. In some cases, the color might differ slightly from the colors of ‘RYOKU NH-13’ recited in the description.

FIG. 1 shows a tree body of ‘RYOKU NH-13’ (photographed date: Jul. 10, 2015; photographed location: NIPPON RYOKUSAN CO., LTD; Matsumoto-City, Nagano-prefecture, Japan).

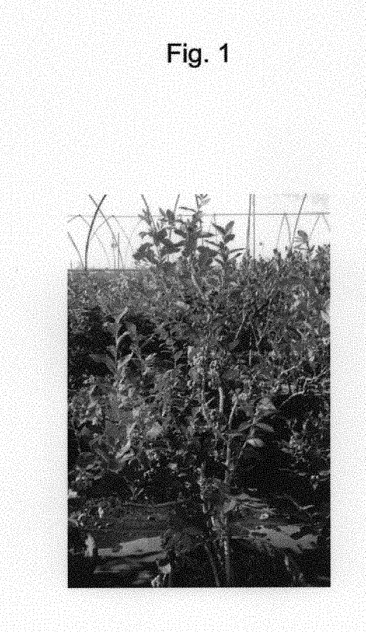

FIG. 2 shows a panoramic view of the cultivation area of ‘RYOKU NH-13’ (photographed date: Sep. 7, 2012; photographed location: same as FIG. 1).

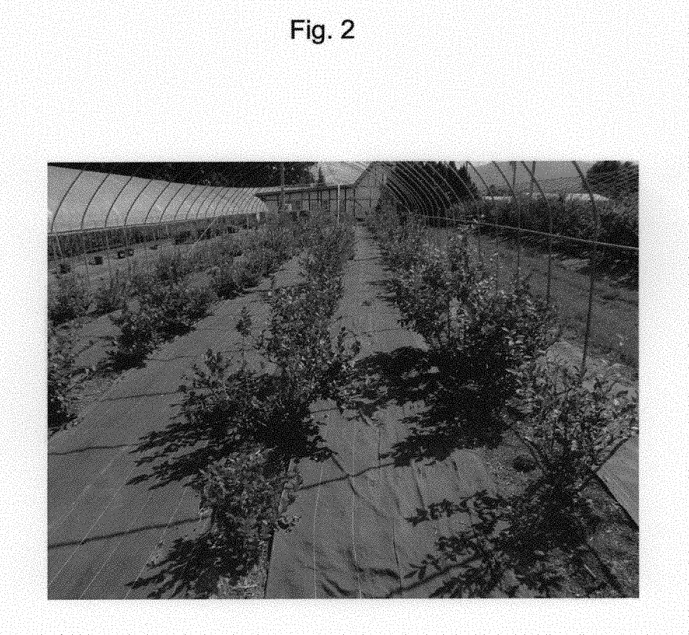

FIG. 3 shows whole flowers of ‘RYOKU NH-13’ (photographed date: May 8, 2015; photographed location: same as FIG. 1).

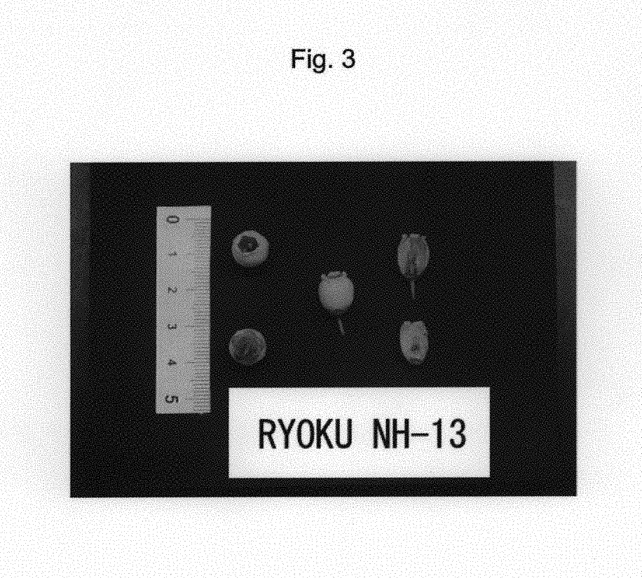

FIG. 4 shows broken-down parts of the flower of ‘RYOKU NH-13’ (photographed date: May 8, 2015; photographed location: same as FIG. 1).

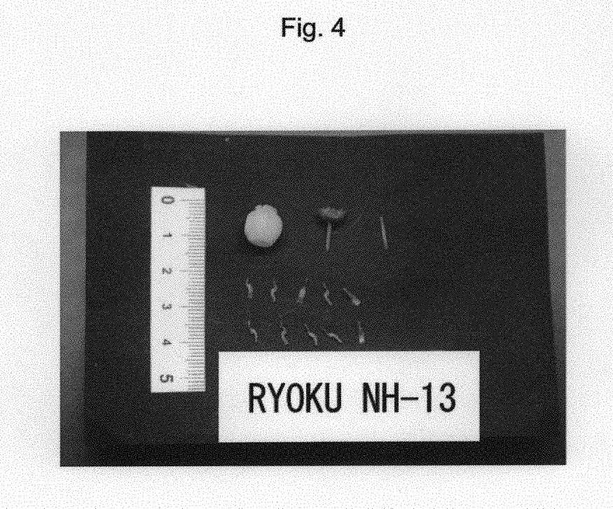

FIG. 5 shows fruits of ‘RYOKU NH-13’ (photographed date: Jul. 14, 2014; photographed location: same as FIG. 1).

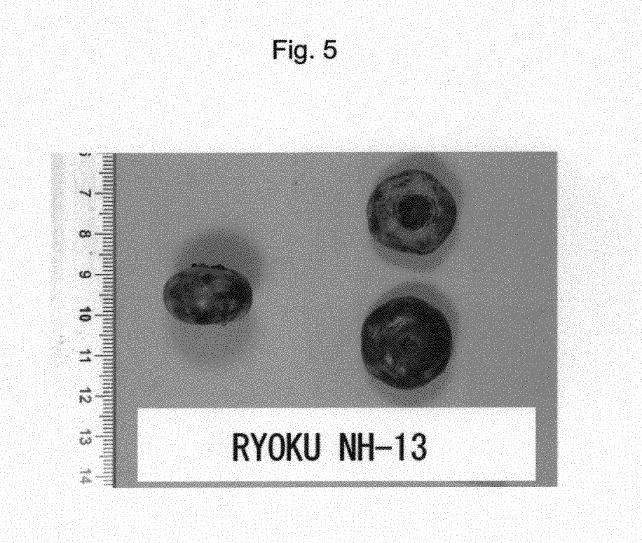

FIG. 6 shows a cross-section of the fruits of ‘RYOKU NH-13’ (photographed date: Jul. 14, 2014; photographed location: same as FIG. 1).

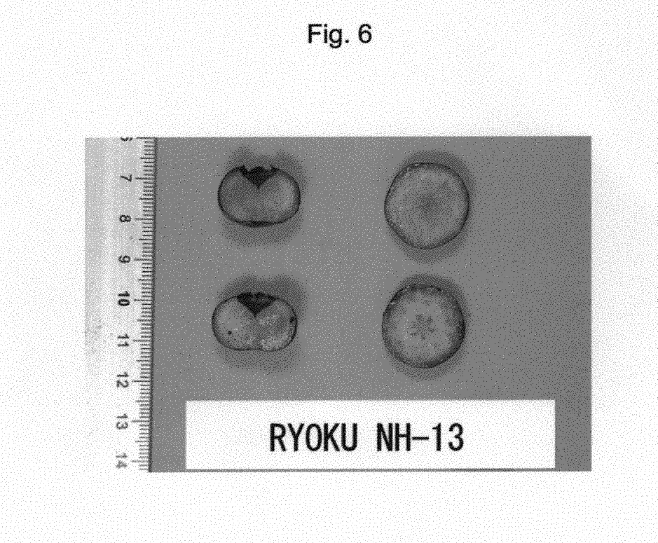

FIG. 7 shows an upper side of the leaves (10 sheets) of ‘RYOKU NH-13’ (photographed date: Jul. 22, 2014; photographed location: same as FIG. 1).

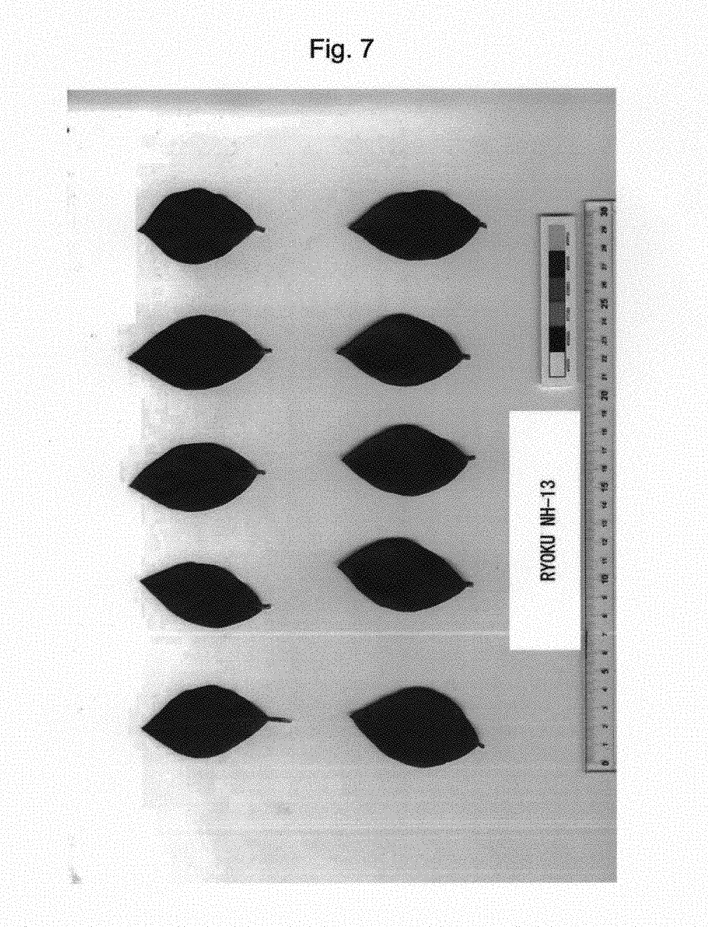

FIG. 8 shows a lower side of the leaves (10 sheets) of ‘RYOKU NH-13’ (photographed date: Jul. 22, 2014; photographed location: same as FIG. 1).

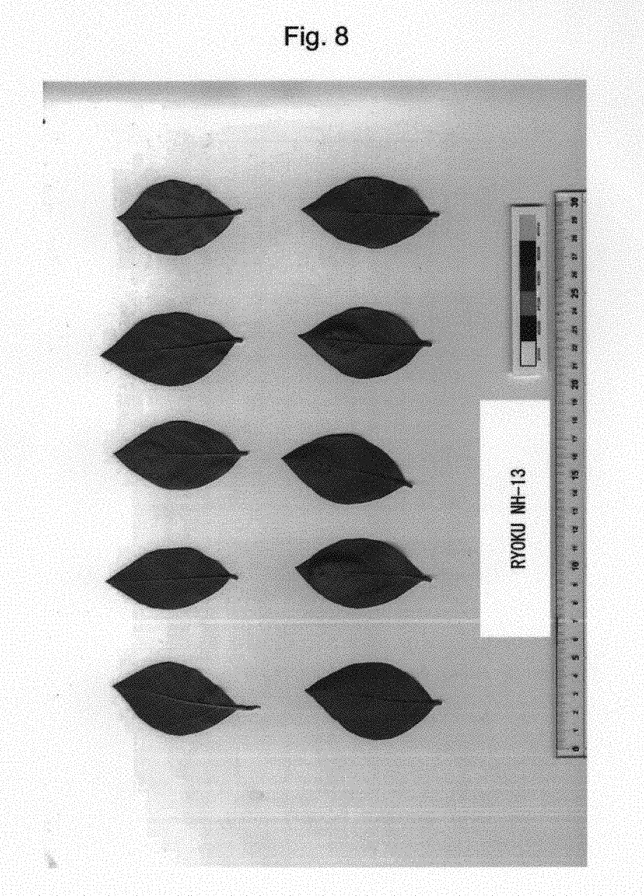

### DETAILED BOTANICAL DESRIPTION

### A. Distinctive Characteristics of ‘RYOKU NY-11’

As described above, ‘RYOKU NH-13’ was obtained by the open pollination of ‘Chandler’, a seed parent (Note: the possible pollen parent might be ‘Blueray’). On Apr. 15, 2003, about 10,000 seeds of ‘Chandler’, which were cultivated in the field of NIPPON RYOKUSAN CO., LTD. (Matsumoto, Nagano, Japan), were seeded in plant seeding trays, and then transplanted to pots. The resulting seedlings (about 3,000) were planted in cultivation pots on May 1, 2004. Fructification of the planted seedlings were started from Jun. 20, 2007 (on Year 3), and about 200 plant individuals were selected based on the characteristics, including larger fruit size, better taste balance of sweetness and acidity, higher yield per plant, earlier ripening time, large and uniform fruits, etc. For the about 200 selected plant individuals, test plots (each including about 20 cuttings per plant individual) were formed, where these plants were asexually propagated by cutting means. During the period from Jul. 20, 2009 to Dec. 25, 2016 and for 3 generations, the plants were propagated and examined for their characteristics based on the growth, yield and fruits quality in each test plot. For 10 test plots, the characteristics of the plants were observed for the period between the flowering time and the ripening time yearly for 5 years, and since neither variant nor off-type plant was observed for the period, the characterization of ‘RYOKU NH-13’ was finished on Dec. 25, 2016 and the breeding was completed.

‘RYOKU NH-13’ is a blueberry clone which can be distinguished from the important blueberry varieties ‘Chandler’ and ‘Blueray’, both of which are widely planted in the Chubu district of Japan, due to its characteristics including a more upright plant growth habit, stronger plant vigor, comparatively early fruit ripening time, comparatively large fruit size, sweeter fruits, better taste balance of sweetness and acidity, better taste of fruits, and smaller stem scar. 31 plants of ‘RYOKU NH-13’ had been propagated by cutting means in Matsumoto, Nagano, Japan, and all the resulting plants were phenotypically indistinguishable from the original plant variety ‘RYOKU NH-13’. In addition, comparing to ‘Chandler’ and ‘Blueray’, the claimed plant ‘RYOKU NH-13’ has a more upright plant growth habit, about 1 week to 10 days earlier fruit ripening time (on average, around July 10 of each year in Matsumoto, Nagano, Japan), sweeter fruits, better taste of fruits, and smaller stem scar when compared to its related variety ‘Chandler’, and has stronger plant vigor, larger fruit size, better taste balance of sweetness and acidity, better taste of fruits, and smaller stem scar when compared to ‘Blueray’ (see Table 1 below).

The following data defining the characteristics of ‘RYOKU NH-13’ were collected from the asexual propagation carried out in Matsumoto, Nagano, Japan. The plant history was taken on a plot of 10 five-year-old plants growing in Matsumoto, Nagano, Japan. ‘RYOKU NH-13’ has not been observed under all possible environmental conditions, and the measurements provide might therefore vary when grown in different environments. Where averages are given, the sample size was 10.

### B. Phenotypic Description of Vaccinium vorymbosum L. (‘RYOKU NH-13’)

Characteristics of ‘RYOKU NH-13’ are further specifically described as follows:
1. Plant:
   Plant vigor.—Strong, where the plant vigor is stronger than ‘Blueray’.
   Plant size.—Large.
   Growth habit.—Upright.
   Plant height.—1.8 m on average for 5-year old plant.
   Plant spread.—1.1 m on average for 5-year old plant.
   Tendency toward evergreenness.—Absent.
   Cold hardiness.—Survived in winter frost (below −10° C.) with minimum damage.
   Ease of propagation.—Propagated by each of dormant wood cutting and softwood stem cutting, where the rooting percentage was greater than 70% and comparable to the other varieties.
2. Trunk and branchea:
   Suckering tendency.—Less sucker as in ‘Chandler’.
   Surface texture (of 6-month-old shoots).—Medium smoothness.
   Surface texture (of 3-year-old and older wood).—Medium smoothness.
   Color of new twigs observed in the field.—Orangish yellow green.
   Internode length.—20.4 mm on average.
3. Leaves:
   Leaf length including petiole.—70.4 mm on average.
   Leaf width of leaf at widest point.—39.2 mm on average.
   Leaf shape.—Elliptic.
   Leaf margin.—Entire.
   Color.—Upper surface: Medium Green.
   Pubescence.—Upper Surface of leaves: Absent. Lower Surface of leaves: Absent. Margins: Absent.
   Timing of vegetative bud burst.—Medium.
4. Flowers:
   Shape.—Campanulate.
   Flowering period.—Mean date of 50% opening of flowers in Matsumoto-City, Nagano-prefecture, Japan is May 3, equivalent to ‘Chandler’.
   Corolla.—Diameter: 9.4 mm on average. Length (from pedicel attachment point to corolla tip excluding the pedicel): 12.0 mm on average. Color: White. Anthocyanin coloration in corolla tube — Medium.
5. Reproductive organs:
   Pollen.—Color: Yellow.
6. Fruit:
   Mean date of 50% harvest in Matsumoto-City, Nagano-prefecture.—July 10.
   Diameter of calyx aperture on mature berry.—7.6 mm on average.
   Size and shape of calyx lobe on mature berry.—Medium size, outcurving, and having deep calyx basin.
   Detachment force for ripe berries (easy, medium, hard).—Easy.
   Fruit cluster density (sparse, medium, dense).—Medium to slightly dense.
   Fruiting type.—On one-year old shoots only.
7. Berry:
   Cluster (tight, medium, loose).—Medium.
   Weight (on well-pruned plants).—3.81 g on average.
   Height.—13.3 mm on average.
   Width.—21.3 mm on average.
   Shape.—Oblate.
   Surface color of mature berries ripe on the plant.—Blue purple.
   Intensity of fruit bloom.—Medium.
   Surface color of ripe berry after polishing.—Medium blue.
   Immature berry color, with bloom.—Light to medium green.
   Immature berry color, without bloom.—Yellow green.
   Flesh color.—Light green.
   Peel color.—Medium blue.
   Pedicel scar.—Medium, 2.54 mm on average.
   Firmness.—Medium.
   Intensity of fruit sweetness.—High, Bx 12.3.
   Intensity of fruit acidity.—High, pH 2.55.
   Texture.—Crispy, juicy, medium seeds.
8. Use: ‘RYOKU NH-13’ produce northern highbush blueberries suitable for fruit-picking farms, fresh fruit markets and processed fruit markets, etc.
9. Resistance to disease, insects, and mites: ‘RYOKU NH-13’ grew vigorously and showed excellent bush survival in the field. It appears to be tolerant to stem blight (Botryosphaeria spp.) and root rot (Phytophthora cinnamoni), with very few young plants dying soon after planting. The response of ‘RYOKU NH-13’ to the various fungal species that cause summer leaf spots is typical of other northern highbush varieties, and fungicide applications may be needed after harvest in order to reduce foliar diseases and to retain leaves until autumn and make maximum flower bud set. Similarly, susceptibility to typical blueberry insect and mite pathogens, such as spotted wing drosophila (Drosophila suzukii), blueberry gall midge (Dasineura oxycoccana) and blueberry bud mite (Acalitus vaccini) is similar to other northern highbush cultivars.

TABLE 1
(Comparison of characteristics among varieties)

| | | | | | | | | | The | | |
|---|---|---|---|---|---|---|---|---|---|---|---|
| | | | | | | | | | present | | |
| | | | | | | | | Standard | variety | | |
| Charact. | UPOV | | | | | | | Variety | RYOKU | Control Varieties | |
| No. | No. | Code | Characteristics | Definition | Method | Class | State | (Ex. Var. ) | NH-13 | Blueray | Chandler |
| 1 | 1 | QN | Plant: vigor | Strength | Observ- | 3 | weak | Bluetta, | 7 | 6 | 7 |
| | (*) | (+) | | of growth | ation | 5 | medium | Meader | | | |
| | | | | level | (a) | 7 | strong | Collins, | | | |
| | | | | of plant | VG | | | Weymouth | | | |
| | | | | | | | | Berkeley, | | | |
| | | | | | | | | Homebell, | | | |
| | | | | | | | | Woodard | | | |
| 2 | | QN | Plant: size | Size of | Observ- | 3 | small | Avonblue, | 7 | 5 | 7 |
| | | | | plant crown | ation | 5 | medium | Bluetta, | | | |
| | | | | | (a) | 7 | large | Flordablue | | | |
| | | | | | VG | | | Bluecrop, | | | |
| | | | | | | | | Earliblue | | | |
| | | | | | | | | Dixi, | | | |
| | | | | | | | | Homebell, | | | |
| | | | | | | | | Tifblue | | | |
| 3 | 2 | QN | Plant: | Whole | Observ- | 1 | upright | Becyblue, | 1 | 1 | 2 |
| | (*) | G | growth habit | shape of | ation | 2 | semi- | Bluechip, | | | |
| | | | | plant without | (a) | 3 | upright | June, | | | |
| | | | | pruning | VG | | spreading | Spartan | | | |
| | | | | during | | | | Bluecrop, | | | |
| | | | | dormant | | | | Lateblue | | | |
| | | | | period | | | | Northland, | | | |
| | | | | | | | | Weymouth | | | |
| 4 | 3 | PQ | One-year-old | Color of | Observ- | 1 | green | Briteblue, | 5 | 5 | 4 |
| | | | shoot: color | middle part | ation | 2 | greenish red | Homebell | | | |
| | | | | of shoot | (a) | 3 | greyish red | Berkeley, | | | |
| | | | | extended | VG | 4 | reddish | Dixi | | | |
| | | | | before | | 5 | yellow | Blueray, | | | |
| | | | | dormant | | 6 | reddish | Darrow, | | | |
| | | | | period | | | brown | Weymouth | | | |
| | | | | | | | dark red | | | | |
| 5 | | QN | One-year-old | Length of | Measure- | 3 | short | | 6 | 5 | 7 |
| | | | shoot: length | middle part | ment mm | 5 | medium | | (212 | (173 | (288 |
| | | | | of shoot | (a) | 7 | long | | mm) | mm) | mm) |
| | | | | extended | VG | | | | | | |
| | | | | before | | | | | | | |
| | | | | dormant | | | | | | | |
| | | | | period | | | | | | | |
| 6 | 4 | QN | One-year-old | Length of | Observ- | 3 | short | Avonblue, | 7 | 3 | 5 |
| | | | shoot: length | internode | ation | 5 | medium | Weymouth | (20.4 | (14.0 | (16.7 |
| | | | of internode | of shoot | (a) | 7 | long | Jersey | mm) | mm) | mm) |
| | | | (upper half) | extended | VG | | | | | | |
| | | | | before | | | | | | | |
| | | | | dormant | | | | | | | |
| | | | | period | | | | | | | |
| | | | | (upper half) | | | | | | | |
| 7 | 5 | QN | Leaf: length | Length | Measure- | 3 | short | | 5 | 5 | 5 |
| | (*) | | | of leaf | ment mm | 5 | medium | | (70.4 | (68.0 | (67.0 |
| | | | | sufficiently | (b) | 7 | long | | mm) | mm) | mm) |
| | | | | expanded | MS/VG | | | | | | |
| 8 | 6 | QN | Leaf: width | Maximum | Measure- | 3 | narrow | | 5 | 5 | 5 |
| | | | | width of | ment mm | 5 | medium | | (39.2 | (31.0 | (36.3 |
| | | | | mature leaf | (b) | 7 | broad | | mm) | mm) | mm) |
| | | | | | MS/VG | | | | | | |
| 9 | 7 | QN | Leaf: ratio | Ratio of leaf | Measure- | 3 | small | | 5 | 5 | 5 |
| | | | length/width | length to | ment | 5 | medium | | (1.80) | (2.20) | (1.85) |
| | | | | maximum | (b) | 7 | large | | | | |
| | | | | width | MS/VG | | | | | | |
| | | | | (leaf length/ | | | | | | | |
| | | | | leaf width) | | | | | | | |
| 10 | 8 | PQ | Leaf: shape | Shape of | Observ- | 1 | lanceolate | Northland | 3 | 3 | 3 |
| | (*) | | | mature leaf | ation | 2 | ovate | Berkeley, | | | |
| | | | | | (b) | 3 | elliptic | Collins, | | | |
| | | | | | VG | 4 | oblong | Coville | | | |
| 11 | | QN | Leaf: shape | Shape of | Observ- | 3 | acute | Weymouth, | 5 | 5 | 5 |
| | | (+) | of tip | tip of | ation | 5 | medium | Woodard | | | |
| | | | | mature leaf | (b) | 7 | obtuse | Earliblue, | | | |
| | | | | | VG | | | Tifblue | | | |
| | | | | | | | | Berkeley, | | | |
| | | | | | | | | Climax, | | | |
| | | | | | | | | Southland | | | |
| 12 | 9 | QL | Leaf: color of | Color of | Observ- | 1 | yellow | Bluechip, | 2 | 2 | 2 |
| | | | upper side | surface of | ation | 2 | green | Bluecrop, | | | |
| | | | | mature leaf | (b) | | | Blueray | | | |
| | | | | | VG | | | | | | |
| 13 | 10 | QN | Only varieties | Intensity | Observ- | 3 | light | | 5 | 5 | 5 |
| | (*) | | with green leaf | of green | ation | 5 | medium | | | | |
| | | | color: | color on | (b) | 7 | dark | | | | |
| | | | Leaf: intensity | surface of | VG | | | | | | |
| | | | of green color | mature leaf | | | | | | | |
| | | | on upper side | | | | | | | | |
| 14 | 11 | QL | Leaf: margin | Type of | Observ- | 1 | entire | | 1 | 1 | 1 |
| | (*) | | | margin of | ation | 2 | serrate | | | | |
| | | | | mature leaf | (b) | | | | | | |
| | | | | | VG | | | | | | |
| 15 | 12 | QN | Flower bud: | Intensity of | Observ- | 3 | weak | | 5 | 7 | 5 |
| | | | anthocyanin | anthocyanin | ation | 5 | medium | | | | |
| | | | coloration | coloration | (a) | 7 | strong | | | | |
| | | | | of flower | VG | | | | | | |
| | | | | bud occurring | | | | | | | |
| | | | | to one year | | | | | | | |
| | | | | old shoot | | | | | | | |
| 16 | 13 | QN | Inflorescence: | Length of | Measure- | 3 | short | | 7 | 5 | 6 |
| | | | length | inflorescence | ment mm | 5 | medium | | (42.0 | (28.7 | (35.1 |
| | | | (excluding | at flower- | (c) | 7 | long | | mm) | mm) | mm) |
| | | | peduncle) | ing time | MS/VG | | | | | | |
| | | | | (excluding | | | | | | | |
| | | | | peduncle) | | | | | | | |
| 17 | 14 | PQ | Flower: shape | Shape of | Observ- | 1 | urceolate | Bluecrop, | 2 | 1-2 | 2-3 |
| | | | of corolla | corolla at | ation | 2 | campan- | Jersey | | | |
| | | | | full bloom | (c) | 3 | ulate | Northblue, | | | |
| | | | | | VG | | cylindrical | Northsky | | | |
| 18 | | PQ | Flower: color | Color of | Observ- | 1 | white | Aliceblue, | 1 | 3 | 1 |
| | | | of corolla | corolla at | ation | 2 | creamy | Bluetta, | | | |
| | | | | full bloom | (c) | 3 | white | Briteblue | | | |
| | | | | | VG | 4 | greenish | Avonblue, | | | |
| | | | | | | | white | Berkeley, | | | |
| | | | | | | | light pink | Bluecrop | | | |
| | | | | | | | | Blueray, | | | |
| | | | | | | | | Collins, | | | |
| | | | | | | | | Coville | | | |
| | | | | | | | | Bluebell, | | | |
| | | | | | | | | Delite, | | | |
| | | | | | | | | Dixi, | | | |
| | | | | | | | | Tifblue | | | |
| 19 | 15 | QN | Flower: size of | Size of | Observ- | 3 | small | | 7 | 5 | 7 |
| | (*) | | corolla tube | corolla tube | ation | 5 | medium | | | | |
| | | | | at full bloom | (c) | 7 | large | | | | |
| | | | | | VG | | | | | | |
| 20 | 16 | QN | Flower: | Intensity of | Obser- | 1 | absent or | | 1 | 3 | 1 |
| | | | anthocyanin | anthocyanin | vation | 3 | very weak | | | | |
| | (*) | | coloration of | coloration | (c) | 5 | weak | | | | |
| | | | corolla tube | on surface of | VG | 7 | medium | | | | |
| | | | | corolla tube | | | strong | | | | |
| 21 | 17 | QL | Flower: ridges | Presence or | Observ- | 1 | absent | Herbert | 1 | 9 | 9 |
| | | | on corolla tube | absence of | ation | 9 | present | Aliceblue, | | | |
| | | | | ridges on | (c) | | | Berkeley, | | | |
| | | | | corolla tube | VG | | | Dixi | | | |
| 22 | 18 | QN | Fruit cluster: | Density of | Observ- | 3 | sparse | Homebell, | 6 | 7 | 7 |
| | | | density | fruit | ation | 5 | medium | Jersey, | | | |
| | | | | per fruit | (d) | 7 | dense | Woodard | | | |
| | | | | cluster | VG | | | Bluechip, | | | |
| | | | | | | | | Bluecrop, | | | |
| | | | | | | | | Bluetta | | | |
| | | | | | | | | Darrow, | | | |
| | | | | | | | | Herbert, | | | |
| | | | | | | | | Patriot | | | |
| 23 | 19 | QN | Unripe fruit: | Intensity of | Observ- | 3 | light | | 4 | 7 | 3 |
| | (*) | | intensity of | green | ation | 5 | medium | | | | |
| | | | green color | color of | VG | 7 | dark | | | | |
| | | | | fruit before | | | | | | | |
| | | | | ripening | | | | | | | |
| 24 | 20 | QN | Fruit: size | Size of fruit | Observ- | 3 | small | Homebell, | 7 | 5 | 7 |
| | (*) | | | at ripening | ation | 5 | medium | June, | (3.81 g) | (2.89 g) | (5.72 g) |
| | | | | | (d) | 7 | large | Northblue | | | |
| | | | | | VG | | | Collins, | | | |
| | | | | | | | | Earliblue | | | |
| | | | | | | | | Berkeley, | | | |
| | | | | | | | | Bluecrop, | | | |
| | | | | | | | | Spartan | | | |
| 25 | 21 | PQ | Fruit: shape in | Shape in | Observ- | 1 | elliptic | Berkeley, | 3 | 3 | 3 |
| | (*) | (+) | longitudinal | longitudinal | ation | 2 | round | Jersey, | | | |
| | | | section | section of | (d) | 3 | oblate | Sharpblue | | | |
| | | | | fruit at | VG | | | Earliblue, | | | |
| | | | | ripening | | | | Halton, | | | |
| | | | | | | | | Woodard | | | |
| 26 | | QN | Fruit: size of scar | Size of stem | Observ- | 3 | small | | 5 | 7 | 7 |
| | | (+) | | scar of | ation | 5 | medium | | (2.54 | (3.80 | (3.98 |
| | | | | mature fruit | (d) | 7 | large | | mm) | mm) | mm) |
| | | | | | VG | | | | | | |
| 27 | | PQ | Fruit: shape of | Shape of | Observ- | 1 | star | | 2 | 2 | 2 |
| | | (+) | calyx cavity | calyx | ation | 2 | circular | | | | |
| | | | | cavity of | (d) | | | | | | |
| | | | | mature fruit | VG | | | | | | |
| 28 | 22 | QN | Fruit: attitude | Attitude of | Observ- | 1 | erect | | 2 | 3 | 3 |
| | | | of sepals | sepals | ation | 2 | erect to | | | | |
| | | | | relative to | (d) | 3 | semi-erect | | | | |
| | | | | mature fruit | VG | 4 | semi-erect | | | | |
| | | | | | | | level | | | | |
| 29 | 23 | QN | Fruit: type of | Direction of | Observ- | 1 | incurving | | 3 | 1 | 3 |
| | | | sepals | curving of | ation | 2 | straight | | | | |
| | | | | sepals | (d) | 3 | reflexed | | | | |
| | | | | | VG | | | | | | |
| 30 | 24 | QN | Fruit: diameter | Diameter of | Observ- | 3 | small | Avonblue, | 7 | 5 | 7 |
| | | | of calyx | calyx | ation | 5 | medium | Bluechip, | (7.60 | (4.40 | (6.46 |
| | | | basin | basin of | (d) | 7 | large | Sharpblue | mm) | mm) | mm) |
| | | | | mature fruit | VG | | | Blueray, | | | |
| | | | | | | | | Woodard | | | |
| | | | | | | | | Coville, | | | |
| | | | | | | | | Darrow, | | | |
| | | | | | | | | Homebell | | | |
| 31 | 25 | QN | Fruit: depth of | Depth of | Observ- | 3 | shallow | Bluecrop, | 7 | 7 | 7 |
| | | | calyx basin | calyx | ation | 5 | medium | Rancocas, | (3.22 | (2.60 | (2.32 |
| | | | | basin of | (d) | 7 | deep | Trfblue | mm) | mm) | mm) |
| | | | | mature fruit | VG | | | Earliblue, | | | |
| | | | | | | | | Jersey | | | |
| | | | | | | | | Blueray, | | | |
| | | | | | | | | Collins, | | | |
| | | | | | | | | Dixi | | | |
| 32 | 26 | QN | Fruit: intensity | Intensity of | Observ- | 1 | very weak | Dixi, | 5 | 5 | 5 |
| | (*) | | of bloom | bloom on | ation | 3 | weak | Herbert, | | | |
| | | | | surface of | (d) | 5 | medium | Sharpblue | | | |
| | | | | mature fruit | VG | 7 | strong | Collins, | | | |
| | | | | | | | | Coville | | | |
| | | | | | | | | Avonblue, | | | |
| | | | | | | | | Bluecrop, | | | |
| | | | | | | | | Tifblue | | | |
| 33 | 27 | PQ | Fruit: color of | Color of skin | Observ- | 1 | light blue | Berkeley, | 2 | 2 | 2 |
| | (*) | G | skin (after | of mature | ation | 2 | medium | Bluechip, | | | |
| | | | removal of | fruit after | (d) | 3 | blue | Tifblue | | | |
| | | | bloom) | removal of | VG | 4 | dark blue | Blueray, | | | |
| | | | | bloom | | | blue red | Jersey, | | | |
| | | | | | | | | June | | | |
| | | | | | | | | Dixi, | | | |
| | | | | | | | | Homebell | | | |
| 34 | 28 | QN | Fruit: | Firmness | Observ- | 3 | soft | Herbert, | | | |
| | | (+) | firmness | of mature | ation | 5 | medium | Homebell, | | | |
| | | | | fruit | (d) | 7 | firm | Spartan | | | |
| | | | | | VG/VS | 9 | very firm | Collins, | | | |
| | | | | | | | | Dixi | 5 | 6 | 5 |
| | | | | | | | | Coville, | | | |
| | | | | | | | | Southland | | | |
| 35 | | PQ | Fruit: color | Color of | Observ- | 1 | white | Berkeley, | 3 | 3 | 3 |
| | | | of flesh | flesh of | ation | 2 | cream | Bluecrop, | | | |
| | | | | mature | (d) | 3 | light green | Blueray | | | |
| | | | | fruit | VG | 4 | light purple | Eadiblue | | | |
| | | | | | | | | Bluechip, | | | |
| | | | | | | | | Lateblue, | | | |
| | | | | | | | | Shatpblue | | | |
| | | | | | | | | Aliceblue, | | | |
| | | | | | | | | Delite, | | | |
| | | | | | | | | Homebell | | | |
| 36 | 29 | QN | Fruit: | Sweetness | Observ- | 3 | low | Avonblue, | 7 | 7 | 6 |
| | (*) | (+) | sweetness | of mature | ation | 5 | medium | Bluechip | (Bx | (Bx | (Bx |
| | | | | fruit | (d) | 7 | high | Berkeley, | 12.3) | 12.6) | 13.0) |
| | | | | | VG | | | Bluetta, | | | |
| | | | | | | | | Spartan | | | |
| | | | | | | | | Aliceblue, | | | |
| | | | | | | | | Bluecrop, | | | |
| | | | | | | | | Blueray | | | |
| 37 | 30 | QN | Fruit: acidity | Acidity of | Observ- | 3 | low | Earliblue, | 7 | 5 | 6 |
| | (*) | (+) | | mature fruit | ation | 5 | medium | Homebell | (pH | (pH | (pH |
| | | | | | (d) | 7 | high | Blueray, | 2.55) | 3.12) | 2.96) |
| | | | | | VG | | | Herbert | | | |
| | | | | | | | | Collins, | | | |
| | | | | | | | | Elliott, | | | |
| | | | | | | | | Lateblue | | | |
| 38 | 31 | QL | Plant: | Shoots to | Observ- | 1 | on one- | | 1 | 1 | 1 |
| | (*) | G | fruiting type | which fruit | ation | 2 | year-old | | | | |
| | | | | adnate | (c) | | shoots only | | | | |
| | | | | | VG | | on one- | | | | |
| | | | | | | | year-old | | | | |
| | | | | | | | and current | | | | |
| | | | | | | | season’ s | | | | |
| | | | | | | | shoots | | | | |
| 39 | | QN | Fruit: | Tendency | Observ- | 3 | less | Earliblue, | 3 | 3 | 3 |
| | | | tendency of | of | ation | 5 | medium | Herbert, | | | |
| | | | cracking | cracking | (d) | 7 | much | Spartan | | | |
| | | | | during | VG | | | Avonblue, | | | |
| | | | | harvest | | | | Berkeley, | | | |
| | | | | season | | | | Bluechip | | | |
| | | | | | | | | Briteblue, | | | |
| | | | | | | | | Climax, | | | |
| | | | | | | | | Darrow | | | |
| 40 | 32 | QN | Time of | Time of | Measure- | 3 | early | Avonblue, | 5 | 5 | 5 |
| | (*) | (+) | vegetative | beginning to | ment | 5 | medium | Beckyblue | April | April | April |
| | | | bud burst | burst first | MG | 7 | late | Sharpblue | 7(2016) | 6(2016) | 8(2016) |
| | | | | vegetative | | | | Darrow, | | | |
| | | | | bud of each | | | | Weymouth | | | |
| | | | | individual | | | | Elliott, | | | |
| | | | | plant | | | | Lateblue | | | |
| 41 | 33 | QN | Time of | Time of 10% | Measure- | 1 | very early | Bluecrop, | 6 | 5 | 6 |
| | (*) | (+) | beginning | flowering | ment | 3 | early | Collins, | April | April | April |
| | | G | of flowering | occurring | MG | 5 | medium | Woodard | 26 | 23 | 27 |
| | | | on one-year- | to one year | | 7 | late | Dixi, | (2016) | (2016) | (2016) |
| | | | old shoot | old shoot | | 9 | very late | Herbert, | | | |
| | | | | | | | | Lateblue | | | |
| 42 | 34 | QN | Varieties which | Time of 10% | Measure- | 3 | early | | — | — | — |
| | (*) | (+) | fruit on one-year- | flowering | ment | 5 | medium | | | | |
| | | G | old shoot and | occurring | MG | 7 | late | | | | |
| | | | current season’ s | to current | | | | | | | |
| | | | shoot: Time | years shoot | | | | | | | |
| | | | of beginning | | | | | | | | |
| | | | of flowering | | | | | | | | |
| | | | on current | | | | | | | | |
| | | | year’s shoot | | | | | | | | |
| 43 | 35 | QN | Time of | Time of | Measure- | 1 | very early | Avonblue, | 5 | 5 | 7 |
| | | (+) | beginning | 10% fruit | ment | 3 | early | Earliblue, | Middle | Middle | Late |
| | | G | of fruit | ripening on | MG | 5 | medium | Weymouth | July | July | July to |
| | | | ripening | one year | | 7 | late | Herbert, | to late | to late | early |
| | | | on one- | old shoot | | 9 | very late | Jersey | July | July | August |
| | | | year-old shoot | | | | | Briteblue, | | | |
| | | | | | | | | Elliott, | | | |
| | | | | | | | | Tifblue | | | |
| 44 | | QN | Period of | Length | Measure- | 3 | short | Darrow, | 5 | 5 | 5 |
| | | | harvest | of harvest | ment | 5 | medium | Northblue | | | |
| | | | | period | MG | 7 | long | Berkeley, | | | |
| | | | | of fruit | | | | Bluecrop | | | |
| | | | | | | | | Briteblue, | | | |
| | | | | | | | | Tifblue, | | | |
| | | | | | | | | Woodard | | | |
| 45 | 36 | QN | Varieties which | Time of | Measure- | 3 | early | | — | — | — |
| | (*) | (+) | fruit on one-year- | 10% fruit | ment | 5 | medium | | | | |
| | | G | old shoot and | ripening | MG | 7 | late | | | | |
| | | | current season’ s | on current | | | | | | | |
| | | | shoot: Time of | years | | | | | | | |
| | | | beginning | shoot | | | | | | | |
| | | | of ripening | | | | | | | | |
| | | | on current | | | | | | | | |
| | | | year’ s shoot | | | | | | | | |

What is claimed is:

1. A new and distinct variety of Vaccinium corymbosum L. plant named ‘RYOKU NH-13’, as described and illustrated herein.
